(19) United States
(12) Patent Application Publication
(10) Pub. No.: US 2025/0057183 A1
(43) Pub. Date: Feb. 20, 2025

(54) COMPOSITIONS COMPRISING BACTERIAL PROTEIN

(71) Applicant: SUPERBREWED FOOD, INC., New Castle, DE (US)

(72) Inventors: Kartheek ANKELLA, Cranston, RI (US); Megan JONES, Durham, NC (US); Aharon EYAL, Jerusalem (IL); Bryan P. TRACY, Wilmington, DE (US); Alon KARPOL, Tel Mond (IL)

(21) Appl. No.: 18/721,896

(22) PCT Filed: Dec. 20, 2022
(86) PCT No.: PCT/IB2022/062552

§ 371 Date: Jun. 20, 2024

Related U.S. Application Data
(60) Provisional application No. 63/292,470, filed on Dec. 22, 2021.
(60) Provisional application No. 63/302,175, filed on Jan. 24, 2022.

(51) Int. Cl. A23J 3/20; A23J 3/04; A23J 3/08; A23J 3/10; A23J 3/22; C12N 1/20

(52) U.S. Cl. CPC A23J 3/20; A23J 3/04; A23J 3/08; A23J 3/10; A23J 3/225; C12N 1/20

(57) ABSTRACT

Provided is a homogeneous composition comprising protein, wherein at least 1 wt % of a total protein content is bacterial protein: and a carrier selected from the group consisting of water, oil and a combination thereof, wherein a total protein content in the composition is at least 25 wt % or wherein the composition comprises at least one animal protein and a total protein content in the composition is at least 5 wt % of the total composition. Further provided are food ingredients and food products comprising the composition.

### CROSS-REFERENCE TO RELATED APPLICATION

The present application gains priority from U.S. Provisional Application Nos. 63/292,470 filed Dec. 22, 2021 and 63/302,175 filed Jan. 24, 2022, both of which are incorporated by reference as if fully set-forth herein.

### FIELD OF THE INVENTION

The present invention, in at least some embodiments, relates to homogeneous compositions comprising protein, wherein at least 1 wt % of a total protein content is bacterial protein; and a carrier selected from the group consisting of water, oil and a combination thereof, wherein a total protein content in the composition is at least 25 wt % or wherein the composition comprises at least one animal protein and a total protein content in the composition is at least 5 wt % of the total composition.

### BACKGROUND OF THE INVENTION

Proteins are molecules essential to the structure and function of all living organisms. Protein sources include meat, casein and whey (milk proteins), gelatin, soybean, yeast, and grains.

Analogues of dairy and meat products, generally based on vegetable proteins, are known in the art and are becoming increasingly in demand by consumers such as those suffering from lactose intolerance or due to ethical considerations regarding exploitation of animals by the dairy and meat industries and environmental impact of animal farming.

Dairy analogues, such as non-dairy cheese analogues, generally based on vegetable proteins, are known in the art and are becoming increasingly in demand by consumers such as those suffering from lactose intolerance or due to ethical considerations regarding exploitation of animals by the dairy industry and environmental impact of dairy farming.

Known milk analogues, such as almond milk, have low protein contents as compared to animal milk, while known cheese and meat analogues commonly have undesirable flavors and/or textures.

### SUMMARY OF THE INVENTION

According to an aspect of some embodiments of the present invention, there is provided a composition comprising
(i) protein, wherein at least 1 wt % of a total protein content is derived from at least one bacterium; and
(ii) a carrier selected from the group consisting of water, oil and a combination thereof,
   wherein said composition is homogeneous, and wherein
   (a) a total protein content is at least 25 wt % of the total composition, or
   (b) the composition further comprises an animal protein and a total protein content is at least 5 wt % of the total composition.

According to a further aspect of some embodiments of the invention, there is provided a food ingredient comprising the composition as disclosed herein.

According to a further aspect of some embodiments of the invention, there is provided a food produce comprising the food ingredient as disclosed herein.

According to a further aspect of some embodiments of the invention, there is provided an analogue of an animal-or plant-derived food product comprising the composition as disclosed herein.

According to an aspect of some embodiments of the present invention, there is provided a method for the production of a composition as disclosed herein.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIG. 1 is a schematic representation of a method in accordance with an embodiment of the present invention.

### DETAILED DESCRIPTION OF THE INVENTION

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used herein, the term “homogenous” refers to a uniform mixture having no visible lumps or unmixed powder or pockets of liquid.

As used herein, the term “food ingredient” refers to an ingestible constituent used in the preparation of a food product. The food ingredient may be present in the food product in modified or unmodified form.

As used herein, the term “food product” refers to any ingestible product, including solid food products, liquid food products (such as beverages) and semi-solid food products.

As used herein, the term “analogue of an animal-derived product” refers to a non-dairy or vegan product having a similar flavor and texture to that of a conventional animal-derived product, such as a meat product, a fish product, or a dairy product. In some embodiments, the analogue of a dairy product is an analogue of a cheese i.e. an analogue of a cheese formed by coagulation of the milk protein casein.

As used herein, the term “analogue of a plant-derived food product” refers to a food product comprising components present in or isolated from a vegetable.

As used herein, the term “animal protein” refers to a protein present in an animal product.

As used herein, the term “casein component” refers to a subtype of casein phosphoproteins i.e. αs1-casein, αs2-casein, β-casein, and κ-casein. Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

The present invention, in at least some embodiments thereof, relates to a composition comprising
(i) protein, wherein at least 1 wt % of a total protein content is derived from at least one bacterium; and
(ii) a carrier selected from the group consisting of water, oil and a combination thereof,
   wherein the composition is homogeneous and wherein
   (a) a total protein content is at least 25 wt % of the total composition, and
   (b) the composition further comprises an animal protein and a total protein content is at least 5 wt % of the total composition.

According to some embodiments, wherein at least one animal product is present, the total protein content is at least 5 wt % of the total composition, such as 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt % or up to 99 wt % of the total composition.

According to some embodiments, the at least one animal protein provides functionality. According to some such embodiments, the at least one animal protein comprises casein and/or egg albumin, wherein the animal protein provides gelation of the composition. According to some such embodiments, the at least one animal protein comprises a protein derived from a connective tissue of an animal, such as collagen and/or elastin, wherein the animal protein provides a meat-like texture to the composition. According to some such embodiments, the at least one protein comprises a protein derived from a muscle of an animal, such as actin and/or myosin, wherein the animal protein provides tissue texture to the composition. According to some such embodiments, the at least one animal protein comprises ovalbumin and/or casein, wherein the animal protein provides foaming of the composition. The functionality of the animal protein in the composition is not affected by the presence of the protein derived from the bacterium.

According to some embodiments, bacterial protein is present in a range of from about 1 wt % to about 90 wt % of the total protein content of the composition, such as about 1%, about 5 wt %, about 10 wt %, about 15 wt %, about 20 wt %, about 25 wt %, about 30 wt %, about 35 wt %, about 40 wt %, 45 wt %, about 50 wt %, about 55 wt %, about 60 wt %, about 65 wt %, about 70 wt %, about 75 wt %, about 80 wt %, about 85 wt % or about 90 wt %.

According to some embodiments, a total protein content in the composition is from at least 25 wt % to about 90 wt %, such as 25 wt %, about 30 wt %, about 35 wt %, about 40 wt %, 45 wt %, about 50 wt %, about 55 wt %, about 60 wt %, about 65 wt %, about 70 wt %, about 75 wt %, about 80 wt %, about 85 wt % or about 90 wt %.

According to an embodiment, the animal protein is casein or at least one casein component.

According to some embodiments, the at least one casein component is selected from the group consisting of αs1-casein, αs2-casein, β-casein, κ-casein and combinations thereof. According to an embodiment, the at least one casein component is one casein component. According to an embodiment, the at least one casein component is two casein components. According to an embodiment, the at least one casein component is three casein components. According to an embodiment, the at least one casein component is four casein components.

According to an embodiment, the one casein component is αs1-casein. According to an embodiment, the one casein component is αs2-casein. According to an embodiment, the one casein component is β-casein. According to an embodiment, the one casein component is κ-casein.

According to an embodiment, the two casein components are αs1-casein and αs2-casein. According to an embodiment, the two casein components are αs1-casein and β-casein. According to an embodiment, the two casein components are αs1-casein and κ-casein. According to an embodiment, the two casein components are αs2-casein and β-casein. According to an embodiment, the two casein components are αs2-casein and κ-casein. According to an embodiment, the two casein components are β-casein and κ-casein.

According to an embodiment, the three casein components are αs1-casein, αs2-casein and β-casein. According to an embodiment, the three casein components are αs1-casein, αs2-casein, and k-casein. According to an embodiment, the three casein components are αs1-casein, β-casein and κ-casein. According to an embodiment, the three casein components are αs2-casein, β-casein and x-casein.

According to an embodiment, the three casein components are αs1-casein, αs2-casein, β-casein and κ-casein.

According to some embodiments, the two, three or four casein components may each be present in the composition in equal amounts. According to some embodiments, the two, three or four casein components may each be present in different amounts. According to some embodiments, two of the three casein components may be present in the same amount and the third casein component may be present in a different amount. According to some embodiments, three of the four casein components may be present in the same amount and the fourth casein component may be present in a different amount. According to some embodiments, a first and second of the four casein components may be present in a same amount and a third and fourth casein component may be present in a same amount, wherein the amount of the first and second casein components is different from the amount of the third and fourth casein components. According to some embodiments, a first and second of the four casein components may be present in a same amount and a third and fourth casein component may be present in amounts which are different from each other and from the amount of the first and second casein components. According to an embodiment, the at least one casein component is a product of fermentation.

According to some embodiments, the at least one casein is derived from an animal. According to some such embodiments, the animal-derived casein may com comprise post-translational modifications, such as one or more phosphorylated phosphorylation sites, one or more disulfide bonds, or glycolysation.

According to some embodiments, the animal protein comprises whey or at least one whey component. According to some such embodiments, the whey component is selected from the group consisting of alpha-lactoglobulin, alpha-lactalbumin, glycomacropeptide, immunoglobulins, bovine serum albumin and combinations thereof. According to some embodiments, the at least one whey component is a product of fermentation.

According to an embodiment, the animal protein comprises an egg protein. According to some such embodiments, the egg protein is selected from the group consisting of low-density lipoprotein, high-density lipoprotein, phosvitin, livetin, ovalbumin, ovotransferrin, ovalbumin, ovoglobulin, ovomucin, ovomucoid, flavoprotein, avidin, lysozyme and combinations thereof.

According to some embodiments, the oil is a vegetable oil. According to some such embodiments, the vegetable oil is selected from the group consisting of coconut oil, corn oil, canola oil, cottonseed oil, palm oil, peanut oil, canola oil, safflower oil, avocado oil, mustard oil, peanut oil, sesame oil, soybean oil, sunflower oil, olive oil and combinations thereof.

According to some embodiments, the oil is a nut oil. According to some such embodiments, the nut oil is selected from the group consisting of almond oil, beech nut oil, Brazil nut oil, cashew oil, macadamia oil, pecan oil, pine nut oil, pistachio oil, walnut oil, pumpkin seed oil and combinations thereof.

According to some embodiments, the bacterial protein is derived from at least one Gram-positive bacterium, such as from one, two, three or more than three Gram-positive bacteria. According to some such embodiments, the Gram-positive bacterium is of the class Clostridia, optionally of the genus Clostridium, further optionally of the species Clostridium tyrobutyricum.

According to some embodiments, the composition further comprises a vitamin selected from the group consisting of folic acid, Vitamin B1 (Thiamine), Vitamin B12 (Cyanocobalamin), Vitamin B2 (Riboflavin), Vitamin B3 (Niacin), Vitamin B5 (Pantothenic), Vitamin B6 (Pyridoxine), Vitamin B7 (Biotin) and combinations thereof. According to some such embodiments, the vitamin is derived from the bacterium from which the protein is derived.

According to some embodiments, the composition further comprises a mineral selected from the group consisting of zinc, manganese, molybdenum, selenium, iron, potassium, copper and combinations thereof. According to some such embodiments, the mineral is derived from the bacterium from which the protein is derived.

According to some embodiments, the bacterial protein comprises a crude protein concentration of at least 80 wt % of a total dry weight of the bacterium, such as 80 wt %, about 85 wt %, about 90 wt %, about 95 wt % or even about 100 wt % of a total dry weight of the bacterium. According to some preferred embodiments, a crude protein content in said bacterium is from about 83 to about 86 wt %.

According to some embodiments, the protein further comprises a plant protein and/or an animal protein. According to some such embodiments, the plant protein is a protein derived from a plant selected from the group consisting of soy, wheat, pea, lentil, beans (including chickpea, fava bean, kidney bean, black bean, pinto bean, lupini bean, mung bean), hemp, peanut, almond and combinations thereof.

According to some embodiments, the animal protein is selected from the group consisting of casein or derivatives thereof (such as micellar casein, sodium caseinate, calcium caseinate, kappa casein and combinations thereof); whey protein and derivatives thereof (such as hydrolyzed whey protein, concentrates and isolates of whey protein); and egg proteins and derivatives thereof (such as ovalbumin, ovotransferrin, ovomucoid and combinations thereof).

According to some embodiments, the carrier comprises water and oil at a water/oil weight/weight ratio of between about 99/1 and about 1/99, such as about 99/1, about 90/1, about 85/1, about 80/1, about 75/1, about 70/1, about 65/1, about 60/1, about 55/1, about 50/1, about 40/1, about 35/1, about 30/1, about 25/1, about 20/1, about 15/1, about 10/1, about 5/1, about 1/1, about 1/5, about 1/10, about 1/15, about 1/20, about 1/25, about 1/30, about 1/35, about 1/40, about 1/45, about 1/50, about 1/55, about 1/60, about 1/65, about 1/70, about 1/75, about 1/80, about 1/85, about 1/90 or about 1/99.

According to some embodiments, a concentration of oil in the carrier is from about 1 to about 60 wt %, such as about 1, about 5, about 10, about 15, about 20, about 25, about 30, about 35, about 40, about 45, about 50, about 55 or about 60 wt %.

According to some embodiments, the carrier comprises unsaturated and saturated oil. According to some such embodiments, the unsaturated/saturated weight/weight ratio is between about 99/1 and about 1/99, such as about 99/1, about 90/1, about 85/1, about 80/1, about 75/1, about 70/1, about 65/1, about 60/1, about 55/1, about 50/1, about 40/1, about 35/1, about 30/1, about 25/1, about 20/1, about 15/1, about 10/1, about 5/1, about 1/1, about 1/5, about 1/10, about 1/15, about 1/20, about 1/25, about 1/30, about 1/35, about 1/40, about 1/45, about 1/50, about 1/55, about 1/60, about 1/65, about 1/70, about 1/75, about 1/80, about 1/85, about 1/90 or about 1/99.

According to a preferred embodiment, the unsaturated/saturated weight/weight ratio is between about 92/8 and about 10/90. According to some embodiments, the unsaturated oil to saturated oil weight/weight ratio is less than about 20/80, less than about 15/85, or even less than about 10/90.

According to some such embodiments, the carrier comprises coconut oil, comprising from about 80 to about 95 wt % saturated oil in the total oil content.

According to some alternative embodiments, the carrier comprises peanut oil, comprising from about 5 to about 15 wt % saturated oil in the total oil content.

According to some embodiments, the composition further comprises a gum, such as a vegetable gum optionally selected from the group consisting of xanthan gum, agar gum, cellulose gum, guar gum, locust bean gum, pectin and combinations thereof. According to some such embodiments, a weight concentration of the gum in the composition is between 0.02% and 5%, more preferably between about 0.03% and 1%, such as about 0.03%, about 0.05%, about 0.1%, about 0.02%, about 0.05%, about 0.1%, about 0.2%, about 0.3%, about 0.4%, about 0.5%, about 0.6%, about 0.7%, about 0.8%, about 0.9% or about 1.0%.

According to some embodiments, the composition further comprises a starch, such as a starch obtained from a plant including potato, corn, pea and the like, which may be provided in a native form or a chemically- and/or enzymatically-modified form. According to some such embodiments, a weight concentration of the starch in the composition is between about 0.05% and 40%, such as about 0.05%, about 0.1%, about 0.05%, about 0.1%, about 0.2%, about 0.3%, about 0.4%, about 0.5%, about 0.6%, about 0.7%, about 0.8%, about 0.9%, about 1%, about 5%, about 10%, about 15%, about 20%, about 25%, about 30%, about 35% or about 40%. According to some embodiments, the starch is first subjected to heating and mixing prior, then added to the composition and further homogenized. Subsequent to homogenization, the final composition is maintained under refrigeration for a duration in the range of 12 hours to 7 days until the desired texture is attained. According to some embodiments, the composition further comprises at least one flavoring agent. According to some embodiments, the flavoring agent is a natural flavoring substance, such as a fruit or vegetable extract. According to some embodiments, the flavoring agent is an artificial flavoring substance or flavor enhancer. According to some embodiments, the flavoring agent is a nature-identical flavoring substance.

According to a further aspect of some embodiments of the present invention, there is provided a food ingredient comprising the composition as disclosed herein.

According to a further aspect of some embodiments of the present invention, there is provided a food product comprising the feed ingredient as disclosed herein. According to some embodiments, the food product is selected from the group consisting of a hard cheese, a spreadable cheese, a nut butter, a sauce, a dip, a marinade, a mayonnaise and optionally further comprises an animal-or plant-derived protein.

According to a further aspect of some embodiments of the present invention, there is provided an analogue of an animal-or plant-derived food product comprising the composition as disclosed herein, wherein a total protein content of the analogue comprises less than about 95 wt % of animal protein, such as less than 95 wt %, less than 90 wt %, less than 85 wt %, less than 80 wt %, less than 75 wt %, less than 70 wt %, less than 65 wt %, less than 60 wt %, less than 55 wt %, less than 50 wt %, less than 45 wt %, less than 40 wt %, less than 35 wt %, less than 30 wt %, less than 25 wt %, less than 20 wt %, less than 15 wt %, less than 10 wt %, less than 5 wt % or even about 0 wt % animal protein.

According to some such embodiments, the analogue is selected from the group consisting of an analogue of a cheese (such as a hard cheese or a spreadable cheese) and an analogue of a nut butter (such as almond butter, cashew butter, hazelnut butter, macadamia nut butter, peanut butter, pecan butter, pistachio butter, walnut butter).

According to some embodiments, the analogue of a cheese further comprises a milk protein, such as casein and/or whey or derivatives thereof. According to an embodiment, said milk protein forms between 1 wt % and 95% of the total protein content of said cheese analogue.

According to some embodiments, the analogue of a cheese is an analogue of a spreadable cheese. According to some such embodiments, the analogue of a spreadable cheese further comprises a plant-derived protein. According to some embodiments, the analogue of a spreadable cheese further comprises a dairy protein.

According to some embodiments, the analogue of a cheese is an analogue of a hard cheese. According to some such embodiments, the analogue of a hard cheese further comprises a plant-derived protein. According to some embodiments, the analogue of a hard cheese comprises a dairy protein.

According to an aspect of some embodiments of the present invention, there is provided a method for the production of a composition as disclosed herein, the method comprising:
(i) providing a protein source, wherein at least 1 wt % of a total protein content of said protein source is bacterial protein;
(ii) providing a carrier selected from the group consisting of water, oil and a combination thereof;
(iii) blending a first fraction of the protein source with the carrier at a protein source to carrier weight/weight ratio of between 0.01 to 0.5 to form a first blend;
(iv) introducing energy into the first blend to form a first homogenized blend;
(v) blending a second fraction of the protein source with the first homogenized blend at protein source to homogenized blend weight/weight ratio of between 0.01 to 0.5 to form a second blend; and
(vi) introducing energy into the second blend to form a second homogenized blend.

According to some embodiments, the first fraction of the protein source is blended with the carrier at a protein source to carrier weight/weight ratio of between about 0.05 to about 0.2, such as about 0.05, about 0.06, about 0.07, about 0.08, about 0.09, about 0.1, about 0.12, about 0.14, about 0.16, about 0.18 or about 0.2.

According to some embodiments, the second fraction of the protein source is blended with the first homogenized blend at a protein source to homogenized blend weight/weight ratio of between about 0.03 to about 0.15, such as about 0.03, about 0.04, about 0.05, about 0.06, about 0.07, about 0.08, about 0.09, about 0.10, about 0.11, about 0.12, about 0.13, about 0.014 or about 0.15.

According to some embodiments, the second fraction of the protein is different from the first fraction of the protein. In some such embodiments the first and second fractions of the protein comprise different proteins (such as a first fraction being a bacterial protein and a second fraction being a plant protein, or vice versa). In some embodiments, the proteins are from the same source, but comprise different protein concentrations.

According to some embodiments, the method further comprises blending a third fraction of the protein source with the second homogenized blend at protein to homogenized blend weight/weight ratio of between 0.01 to 0.5 to form a third blend; and introducing energy into the third blend to form a third homogenized blend. According to some such embodiments, the third fraction of the protein source is blended with the second homogenized blend at a protein source to homogenized blend weight/weight ratio of between about 0.03 to about 0.15, such as about 0.03, about 0.04, about 0.05, about 0.06, about 0.07, about 0.08, about 0.09, about 0.10, about 0.11, about 0.12, about 0.13, about 0.014 or about 0.15.

According to some embodiments, the third fraction of the protein is different from the second fraction of the protein and/or from the first fraction of the protein. In some such embodiments the first and/or second and/or third fractions of the protein comprise different proteins. In some embodiments, the proteins are from the same source, but comprise different protein concentrations.

According to some embodiments, the method further comprises blending a fourth fraction of the protein source with the third homogenized blend at protein to homogenized blend weight/weight ratio of between 0.01 to 0.5 to form a fourth blend; and introducing energy into the fourth blend to form a fourth homogenized blend. According to some such embodiments, the fourth fraction of the protein source is blended with the third homogenized blend at a protein source to homogenized blend weight/weight ratio of between about 0.03 to about 0.15, such as about 0.03, about 0.04, about 0.05, about 0.06, about 0.07, about 0.08, about 0.09, about 0.10, about 0.11, about 0.12, about 0.13, about 0.014 or about 0.15.

According to some embodiments, the fourth fraction of the protein is different form the third fraction of the protein and/or the second fraction of the protein and/or from the first fraction of the protein. In some such embodiments the first and/or second and/or third and/or fourth fractions of the protein comprise different proteins. In some embodiments, the proteins are from the same source, but comprise different protein concentrations.

According to some embodiments, the method further comprises adding at least one selected from the group consisting of an oil, a gum, a starch, a fat, a divalent cation and a buffering agent or a combination thereof to at least one of the first blend, the first homogenized blend, the second blend, the second homogenized blend, the third blend, the third homogenized blend, the fourth blend, and the fourth homogenized blend.

According to some embodiments, the method further comprises blending a fifth fraction of the protein with the fourth homogenized blend at a protein to homogenized blend weight/weight ratio of between 0.01 and 0.5 to form a fifth blend; and introducing energy into the fifth blend to form a fifth homogenized blend. and introducing energy into the fourth blend to form a fourth homogenized blend. According to some such embodiments, the fifth fraction of the protein source is blended with the fourth homogenized blend at a protein source to homogenized blend weight/weight ratio of between about 0.03 to about 0.15, such as about 0.03, about 0.04, about 0.05, about 0.06, about 0.07, about 0.08, about 0.09, about 0.10, about 0.11, about 0.12, about 0.13, about 0.014 or about 0.15.

According to some embodiments, the fifth fraction of the protein is different from the fourth fraction of the protein and/or the third fraction of the protein and/or the second fraction of the protein and/or from the first fraction of the protein. In some such embodiments the first and/or second and/or third and/or fourth and/or fifth fractions of the protein comprise different proteins. In some embodiments, the proteins are from the same source, but comprise different protein concentrations.

According to some embodiments, the method further comprises adding at least one selected from the group consisting of a fat, an oil, a gum, a starch, a divalent cation and a buffering agent or a combination thereof to at least one of the first blend, the first homogenized blend, the second blend, the second homogenized blend, the third blend, the third homogenized blend, the fourth blend, the fourth homogenized blend, the fifth blend, the fifth homogenized blend, and any additional blends or homogenized blends.

According to some embodiments wherein a fat is added, the fat is preferably added in the first step of homogenization.

According to some embodiments wherein a starch is added, the starch is preferably cooked prior to blending of the first portion of the protein with the carrier. According to some embodiments, the starch is cooked at a temperature of from about 60°° C. to about 93° C. for a duration of from about 2 to about 10 minutes.

According to some embodiments wherein a gum is added, the gum is cooked prior to blending of the first portion of the protein with the carrier. Alternatively, according to some embodiments, no cooking stage is carried out prior to blending of the first portion of the protein with the carrier.

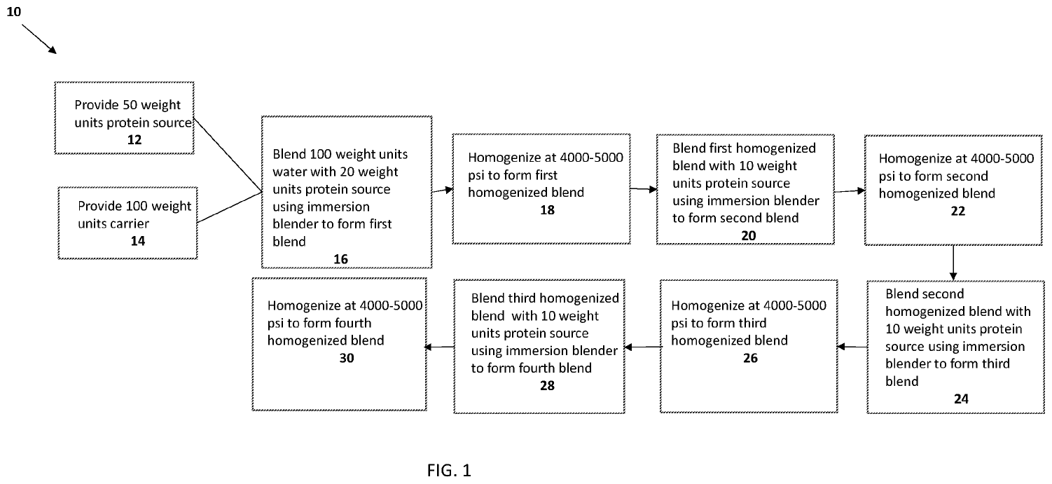

An exemplary process 10 for the preparation of a 1:0.425 water:protein composition in accordance with the principles of the present invention is schematically illustrated in FIG. 1.

According to process 10, there is provided 50 weight units of a protein source comprising 85 wt % protein content (step 12) and 100 weight units water as carrier (step 14).

A first fraction (20 weight units) of the protein source is blended with the 100 weight units water using an immersion blender (step 16) to provide 120 weight units of a first blend. Energy is introduced into the first blend (step 18) by high-pressure homogenization in the range of from about 4000 to about 5000 psi to form a first homogenized blend.

A second blend is then formed (step 20) by blending the first homogenized blend with a second fraction of the protein source (10 weight units) using an immersion blender to provide 130 weight units of the second blend. Energy is introduced into the second blend (step 22) by high-pressure homogenization in the range of from about 4000 to about 5000 psi to form a second homogenized blend.

A third blend is then formed (step 24) by blending the second homogenized blend with a third fraction of the protein source (10 weight units) using an immersion blender to provide 140 weight units of the third blend. Energy is introduced into the third blend (step 26) by high-pressure homogenization in the range of from about 4000 to about 5000 psi to form a third homogenized blend.

A fourth blend is then formed step 28) by blending the third homogenized blend with a fourth fraction (10 weight units) using an immersion blender to provide 150 weight units of the fourth blend. Energy is introduced into the fourth blend (step 30) by high-pressure homogenization in the range of from about 4000 to about 5000 psi to form a fourth homogenized blend 30 as a final product.

Fourth homogenized blend 30 comprises 50 weight units of protein source, the protein source comprising 85 wt % protein, such that fourth homogenized blend 30 has a protein content of 28.3% w/w of the total blend 30.

1. A composition comprising
(i) protein, wherein at least 1 wt % of a total protein content is derived from at least one bacterium; and
(ii) a carrier selected from the group consisting of water, oil and a combination thereof,
wherein said composition is homogeneous, and wherein
(a) a total protein content is at least 25 wt % of the total composition, or
(b) said composition further comprises an animal protein and a total protein content is at least 5 wt % of the total composition.

2. The composition of claim 1, wherein said animal protein comprises casein or at least one casein component.

3. The composition of claim 2, wherein said casein component is selected from the group consisting of αs1-casein, αs2-casein, β-casein, κ-casein and combinations thereof, optionally wherein said at least one casein component is a product of fermentation.

4. (canceled)

5. The composition of claim 1, wherein said animal protein comprises whey or at least one whey component, wherein said whey component is optionally selected from the group consisting of alpha-lactoglobulin, alpha-lactalbumin, glycomacropeptide, immunoglobulins, bovine serum albumin and combinations thereof.

6-7. (canceled)

8. The composition of claim 1, wherein said animal protein comprises an egg protein, optionally selected from the group consisting of albumin, low-density lipoprotein, high-density lipoprotein, phosvitin, livetin, ovotransferrin, ovalbumin, ovoglobulin, ovomucin, ovomucoid, flavoprotein, avidin, lysozyme and combinations thereof.

9. (canceled)

10. The composition of claim 1, wherein said animal protein is a protein derived from a connective tissue or a muscle of an animal, optionally selected from the group consisting of collagen, elastin, actin, myosin or a combination thereof.

11-13. (canceled)

14. The composition of claim 1, wherein said at least one bacterium is a Gram-positive bacterium, optionally of the class Clostridia.

15. The composition of claim 14, wherein said bacterium comprises Clostridium tyrobutyricum.

16. The composition of claim 1, wherein said bacterium comprises a crude protein concentration of at least 80 wt % of a total dry weight of said bacterium, optionally from about 83 wt % to about 86 wt %.

17. The composition of claim 1, wherein said bacterium comprise a vitamin selected from the group consisting of folic acid, Vitamin B1, Vitamin B12, Vitamin B2, Vitamin B3, Vitamin B5, Vitamin B6, Vitamin B7 and combinations thereof and/or a mineral selected from the group consisting of zinc, manganese, molybdenum, selenium, iron, potassium, copper and combinations thereof.

18. The composition of claim 1, wherein said protein further comprises a plant protein and/or an animal protein.

19. The composition of claim 1, wherein said carrier comprises water and oil at a water to oil weight/weight ratio of between 99/1 and 1/99, optionally wherein a concentration of oil in said carrier is from about 1 to about 60 wt %.

20. (canceled)

21. The composition of claim 1, wherein said carrier comprises unsaturated oil and saturated oil at an unsaturated oil to saturated oil weight/weight ratio of between 99/1 and 1/99, optionally wherein said unsaturated oil to saturated oil weight/weight ratio is between 92/8 and 10/90.

22. (canceled)

23. The composition of claim 21, wherein said unsaturated oil to saturated oil weight/weight ratio is less than about 20/80.

24. The composition of claim 1, further comprising a gum, optionally a vegetable gum selected from the group consisting of xanthan gum, agar gum, cellulose gum, guar gum, locust bean gum, pectin and combinations thereof, further optionally wherein a weight concentration of said gum is between 0.1% and 15%.

25-26. (canceled)

27. The composition of claim 1, further comprising a starch, optionally a vegetable-derived starch selected from the group consisting of potato starch, corn starch, pea starch and combinations thereof, further optionally wherein said starch is a chemically- and/or enzymatically-modified starch.

28-30. (canceled)

31. A food ingredient comprising the composition of claim 1 any one of the preceding claims.

32. A food product comprising the food ingredient of claim 31.

33. (canceled)

34. An analogue of an animal-or plant-derived food product comprising the composition of claim 1, wherein a total protein content of said analogue comprises less than 95% wt of animal protein.

35. (canceled)

36. A method for the production of the composition of claim 1, comprising
(i) providing a protein source;
(ii) providing said carrier;
(iii) blending a first fraction of said protein source with said carrier at a protein to carrier weight/weight ratio of between 0.01 and 0.5 to form a first blend;
(iv) introducing energy into said first blend to form a first homogenized blend;
(v) blending a second fraction of said protein source with said first homogenized blend at protein to homogenized blend weight/weight ratio of between 0.01 and 0.5 to form a second blend; and
(vi) introducing energy into said second blend to form a second homogenized blend.

37-41. (canceled)
